(19) United States
(12) Patent Application Publication
(10) Pub. No.: US 2024/0120071 A1
(43) Pub. Date: Apr. 11, 2024

(54) QUANTIFYING AND VISUALIZING CHANGES OVER TIME TO HEALTH AND WELLNESS

(71) Applicant: LoveMyDelta Inc., Philadelphia, PA (US)

(72) Inventors: James M. Smartt, Jr., Philadelphia, PA (US); Bryan Allan Comstock, Kenmore, WA (US); Navdeep S. Dhillon, Seattle, WA (US); Jason David Kelly, Boston, MA (US); David S. Spencer, Seattle, WA (US); Carsten Tusk, Des Moines, WA (US)

(73) Assignee: LoveMyDelta Inc., Philadelphia, PA (US)

(21) Appl. No.: 18/275,129

(22) PCT Filed: Feb. 2, 2022
(86) PCT No.: PCT/US2022/014959

§ 371 Date: Jul. 31, 2023

Related U.S. Application Data
(60) Provisional application No. 63/145,463, filed on Feb. 3, 2021.

(51) Int. Cl. G16H 30/40; G06Q 50/00

(52) U.S. Cl. CPC G16H 30/40; G06Q 50/01

(57) ABSTRACT

Methods, systems, and techniques for providing visual expertise to objectively measure, evaluate, and visualize aesthetic change are provided. Example embodiments provide an Aesthetic Delta Measurement System (“ADMS”), which enables users to objectively measure and visualize aesthetic health and wellness and treatment outcomes and to continuously supplement a knowledge repository of objective aesthetic data based upon a combination of automated machine learning and surveyed human input data. The ADMS provides a labeling platform for labeling aesthetic health and wellness over large populations of individuals and a personal analysis application for viewing an individuals aesthetic changes over time. The ADMS provides labeling of images using guides with corresponding discrete scalar values or using pairwise comparison techniques. It also accommodates dynamic acquisition of images and is able to adjust scoring as appropriate to accommodate this dynamic data using a dynamic ELO ranking algorithm to generate data sets for machine learning purposes.

### CROSS-REFERENCE TO RELATED APPLICATIONS

This application is claims the benefit of U.S. Provisional Patent Application No. 63/145,463, entitled “METHOD AND SYSTEM FOR QUANTIFYING AND VISUALIZING CHANGES OVER TIME TO AESTHETIC HEALTH AND WELLNESS,” filed Feb. 3, 2021, which is incorporated herein by reference in its entirety.

### TECHNICAL FIELD

The present disclosure relates to methods, techniques, and systems for quantifying and visualized changes to aesthetic health and wellness and, in particular, to methods, techniques, and systems for scoring and visualizing changes to aesthetic appearance over time.

### BACKGROUND

Aesthetic medicine has been plagued by a difficultly in objectively assessing the effectiveness of procedures such as plastic surgery, chemical injections, and the like to improve aesthetic health and wellness. Visual assessments generally are both difficult to quantify and difficult to view over a period of time. As well, general and specific group population data is lacking and there is no concept of a “norm” to compare an individual's results to larger and/or specific populations, such as based upon geolocation, ethnicity, etc. For example, an individual can appreciate visual changes in his/her own body and there are criteria that can be used to evaluate such change, such as different scales used for facial aging (e.g., wrinkles, sagging skin, etc.). However, observations at a more macro level—relative to larger groups of individuals are not available. Moreover different professionals looking at a same visual image might conclude differently. Existing scales used to evaluate aesthetic visual images are largely proprietary and do not denote a common currency that can be used for validation of and by professionals. In addition, data privacy and HIPAA concerns limited the ability of data to be shared without restriction.

Thus, potential consumers engage in such services typically based upon advertising of such services—professional or otherwise. Once in a provider's office, the potential consumer can sometimes decide to engage in such services based upon the individual's assessment of “before” and “after” images of others having undergone similar procedures and based upon proprietary software geared to present simulations of the effect of such procedures on that individual customer. The customer has no way to easily measure the effectiveness of such a procedure once performed, let alone, months or even years subsequent to performance the procedure.

For example, visual works of art are generally involve a subjective assessment as to whether one is “good” or “bad.” Although there may be objective metrics as to the production of the art piece that can be used to characterize it (e.g., quality of brushstrokes, light, realism, balance of color, negative space in a painting), ultimately a judgement of whether a particular person likes or dislikes a particular piece of art is highly subjective and involves both a logical and emotional decision.

Aesthetic health and wellness is treated similarly. Specifically, visual knowledge of procedure outcomes is scarce and thus there is little way to gain more statistically significant objective data both before and after aesthetic procedures are performed.

### BRIEF DESCRIPTION OF THE DRAWINGS

The patent or application file contains at least one drawing executed in color. Copies of this patent or patent application publication with color drawings will be provided by the Office upon request and payment of any necessary fee.

FIG. 1 is an example block diagram of an environment for practicing the Aesthetic Delta Measurement System.

FIG. 2 illustrates screen displays of several available front end user interfaces for interacting with an example Aesthetic Delta Measurement System.

FIGS. 3A-3I are example screen displays and other illustrations that show a web based portal, an aesthetic visualization portal, for interacting with an example Aesthetic Delta Measurement System to generate crowd sourced labeled visual image data and to generate physician assisted ground truth data.

FIGS. 4A-4J are example screen displays that illustrate an administration portal to the web based aesthetic visualization portal used for crowd sourced data

FIGS. 5A-5Q are screen displays from an example Aesthetic Delta Measurement System illustrative of a personal analysis application for viewing an individual's aesthetic changes over time.

FIG. 6 illustrates the different bounding rectangles provided in Aesthetic Delta Measurement System client applications.

FIG. 7 illustrates an example ranking of forehead lines on a discrete scale as produced by the trained Aesthetic Delta Measurement System ML model.

FIG. 8 is an example chart illustrating the quintiles and results used for ELO scoring of facial aging in women.

FIG. 9 illustrates a power curve used to adjust the ELO ranking algorithm K-coefficient.

FIG. 10 is an example block diagram of a computing system for practicing embodiments of an example Aesthetic Delta Measurement System including example components.

### DETAILED DESCRIPTION

Embodiments described herein provide enhanced computer- and network-based methods, techniques, and systems for providing visual expertise to objectively measure, evaluate, and visualize aesthetic change. Example embodiments provide an Aesthetic Delta Measurement System (“ADMS”), which enables users to objectively measure and visualize aesthetic health and wellness and treatment outcomes and to continuously supplement a knowledge repository of objective aesthetic data based upon a combination of automated machine learning and surveyed human input data. In this manner aesthetic “knowledge” is garnered and accumulated at both an individual and at larger population levels.

In a first example embodiment, the ADMS provides a labeling platform for labeling aesthetic health and wellness over large populations of individuals and a personal analysis application for viewing an individual's aesthetic changes over time. The labeling platform, currently implemented as software-as-a-service accessible from a web portal, allows objective scoring and ranking of a wide swatch of images reflecting aesthetic health and wellness over vast populations using a combination of machine learning and crowd sourcing. The accumulated, scored, and/or ranked visual image data (annotated or labeled data) are forwarded to a backend computing system for further use in machine learning and analysis. This data can be used, for example, as training, validation, or test data to a machine learning model to classify and predict aesthetic outcomes. The personal analysis application allows objective assessment and evaluation of an individual's aesthetic health and wellness at an instant (e.g., current time) and over a period of time. In one example, both static and dynamic image capture of poses of facial features are collected and visualizations presented and labeled with associated objective assessments. In addition, the visual knowledge (collected, assessed, annotated/labeled data) can be forwarded to the backend computing system configured to apply machine learning to classify, objectively assess the data and/or to supply further training, validation, or test data.

In addition, the ADMS accommodates dynamic acquisition of images and is able to adjust scoring as appropriate to accommodate this dynamic data using a dynamic ranking algorithm to generate data sets for machine learning purposes (e.g., training, test, and validation data). This system is also able to remove garbage responses and maintain HIPAA compliance.

Data acquisition may come from a variety of sources, including physicians, aesthetic wellness and health providers, individuals, and crowd sourced survey data. The data automatically and anonymously collected may be used to adjust manually curated ground truth data for machine learning purposes. For example, the data whether crowd sourced or based upon personal data from procedures can be used for active learning of the ADMS; that is, to further train the machine learning models as more data is accumulated. Thus, the machine learning is enhanced through a combination of data sourcing and guiding annotation to provide model improvements over time. In the example ADMS environments described, data may be acquired by a combination of pairwise comparison and guided scale visual recognition. Other techniques are contemplated.

Although the techniques of the ADMS are being described relative to aesthetic health and wellness, they are generally applicable to the objective measurement, assessment, evaluation, and visualization of any type of image, regardless of content, and provide a may to objectively assess and evaluate visual content in a statistically significant manner. Such images may be still images or videos. Thus, it is to be understood that the examples described herein can be applied to art, photography, industrial drawings, and the like, or to any content that can be rated and compared to an objective metric, such as a scale of severity, presence or not of certain characteristics or features, use of different colors, and the like.

Also, although certain terms are used primarily herein, other terms could be used interchangeably to yield equivalent embodiments and examples. In addition, terms may have alternate spellings which may or may not be explicitly mentioned, and all such variations of terms are intended to be included.

Example embodiments described herein provide applications, tools, data structures and other support to implement an Aesthetic Delta Measurement System to provide statistically significant objective assessment of aesthetic health and wellness data and a knowledge repository for same both at an individual level and a larger group level. Other embodiments of the described techniques may be used for other purposes, including for example, for determining a valuation of artwork, success/failure of application of procedures that result in visual change, and the like. In the following description, numerous specific details are set forth, such as data formats and code sequences, etc., in order to provide a thorough understanding of the described techniques. The embodiments described also can be practiced without some of the specific details described herein, or with other specific details, such as changes with respect to the ordering of the logic, different logic, etc. Thus, the scope of the techniques and/or functions described are not limited by the particular order, selection, or decomposition of aspects described with reference to any particular routine, module, component, and the like.

With respect to aesthetic health and wellness data, an example ADMS is described to address objective assessment and visualization of two examples relative to human aging facial features, namely the assessment of skin aging and wrinkles in the glabellar and forehead regions of the face. Similar techniques may be applied to other bodily areas such as the lip, chin and jowl area, thighs, buttocks, and the like. Aesthetic measurement and evaluation of human beings is particularly difficult because each individual is unique.

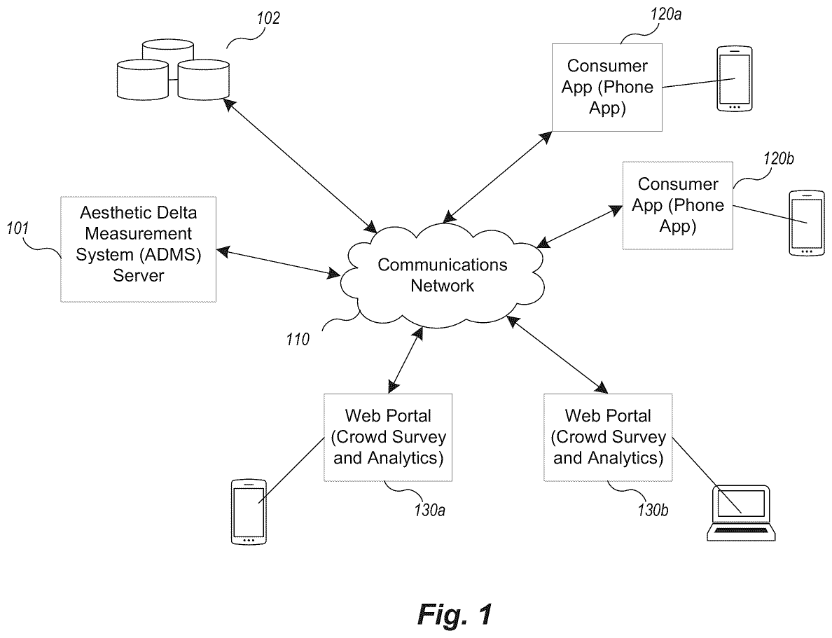

FIG. 1 is an example block diagram of an environment for practicing the Aesthetic Delta Measurement System. In FIG. 1, there is an Aesthetic Delta Measurement System (ADMS) server 101, which communicates to one or more data repositories 102 for storing machine learning and other image data and to one or more front end user interfaces 120 and 130 through communications network 110. The user interfaces 120 and 103 may be mobile or wired applications and may communicate with one or more user participants using a variety of devices including phones, smart personal devices, laptops, computers, and the like. One example ADMS includes a web portal 130a and 130b for facilitating the acquisition of population level labeling of images using aesthetic providers (such as physicians) and using crowd sourced survey techniques. It also includes a consumer application 120a and 120b (e.g., a phone application) that allows an individual to visualize and measure his/her own aesthetic features along and provide the acquisition of individual level labeling of images over time. In some example ADMS environments, the consumer application 120a-b also facilitates the acquisition of population level labeling.

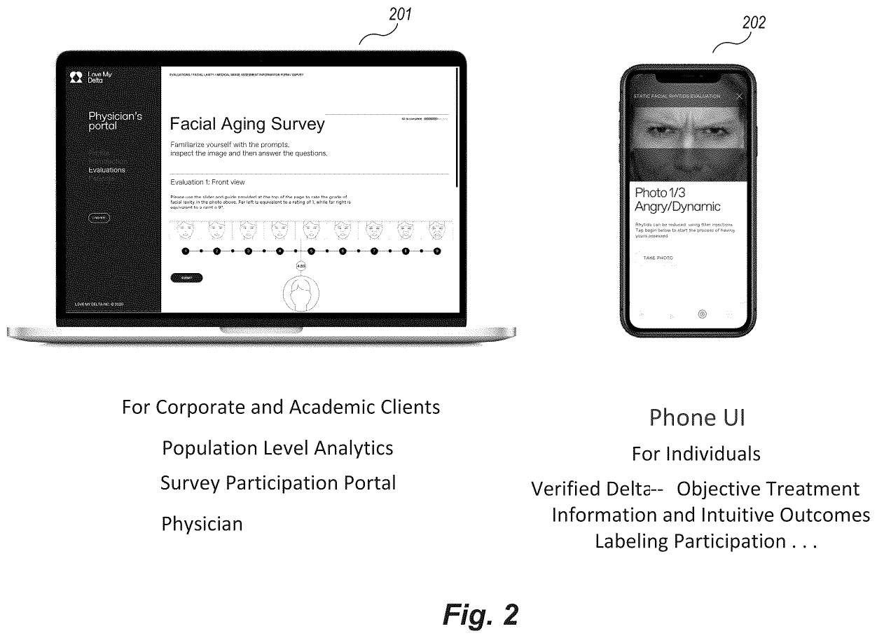

FIG. 2 illustrates screen displays of several available front end user interfaces for interacting with an example Aesthetic Delta Measurement System. Currently two interfaces to the ADMS are available in an example implementation called “Love My Delta” for collecting and visualizing objective aesthetic data. This is but one example of the types of different interfaces that can be configured to interact with ADMS server 101 in FIG. 1. In a first example embodiment, the ADMS provides a labeling platform for labeling aesthetic health and wellness over large populations of individuals, client application (web portal interface) 201, and a personal analysis application for viewing an individual's aesthetic changes over time, a phone application (consumer app) 202.

Web portal 201 is targeted for physicians to obtain ground truth data for the ADMS machine learning capabilities and for obtaining other training, validation, and test data from physicians and from “surveys” available from crowd sourcing technology such as through AMAZON's Mechanical Turk Application Programming Interfaces (API). Phone user interface 202 is directed to providing analysis tools for information regarding an individual's aesthetic features currently and over time. It also provides another manner for obtaining objective labeling of aesthetic images which are forwarded to the ADMS server 101 for machine learning purposes.

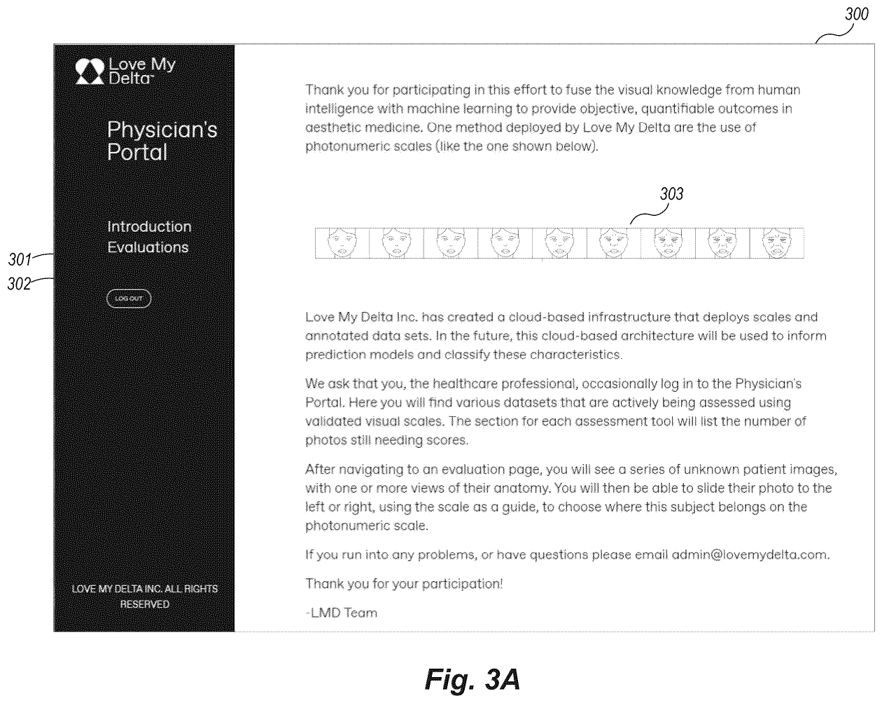

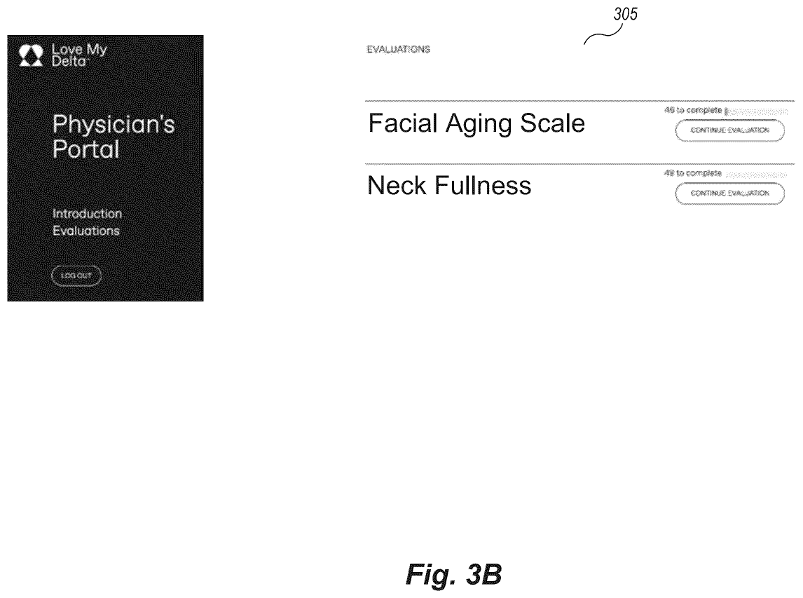

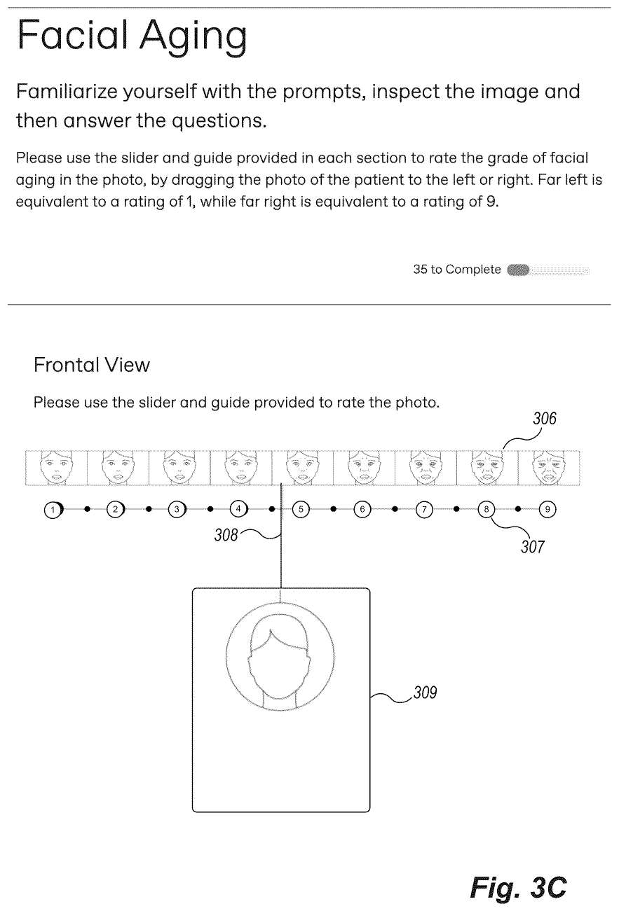

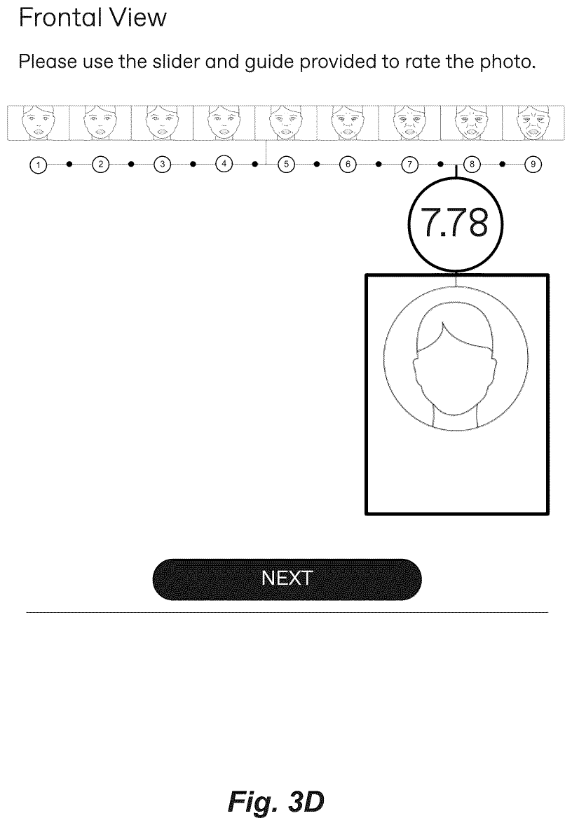

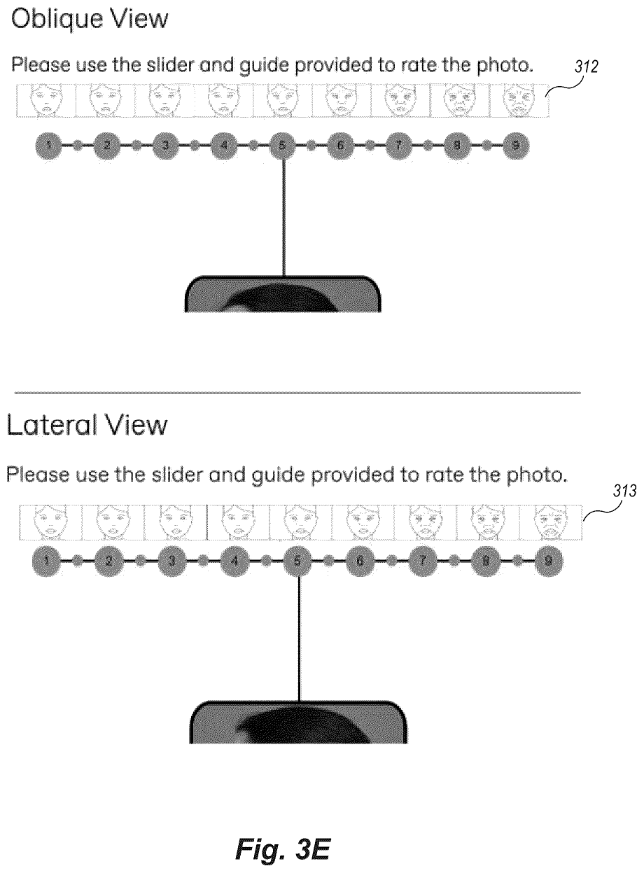

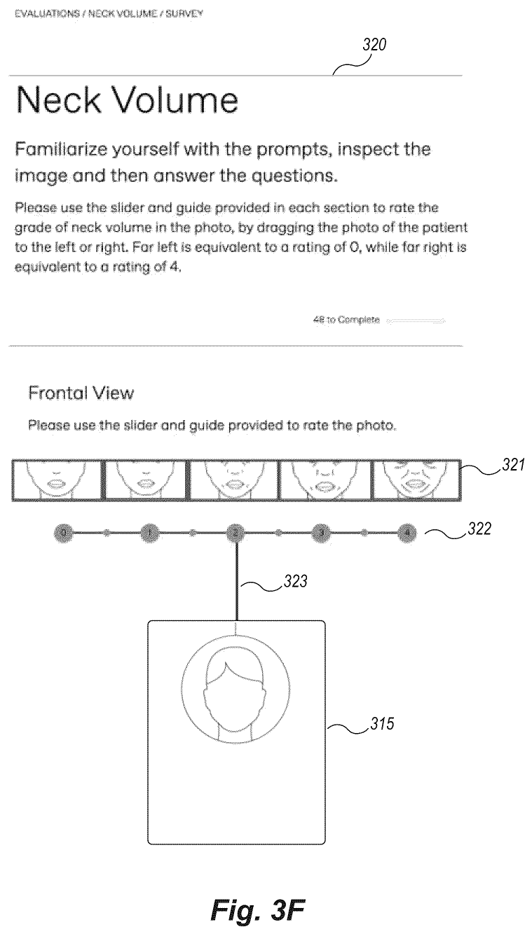

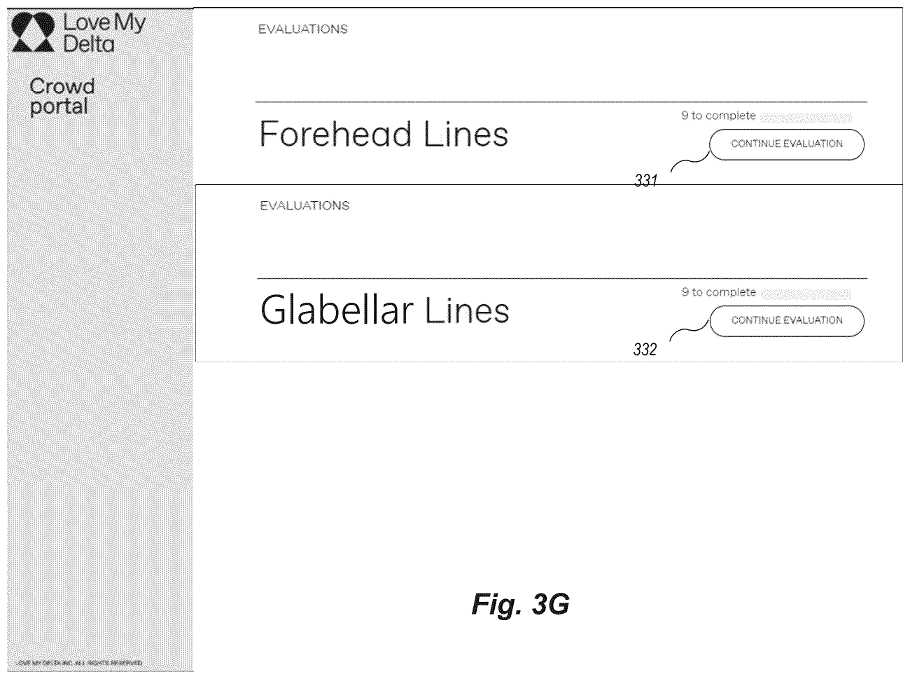

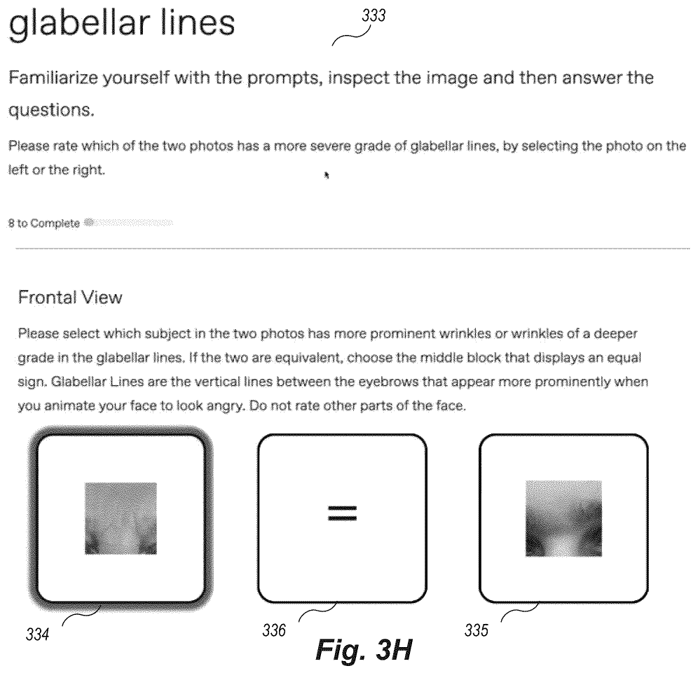

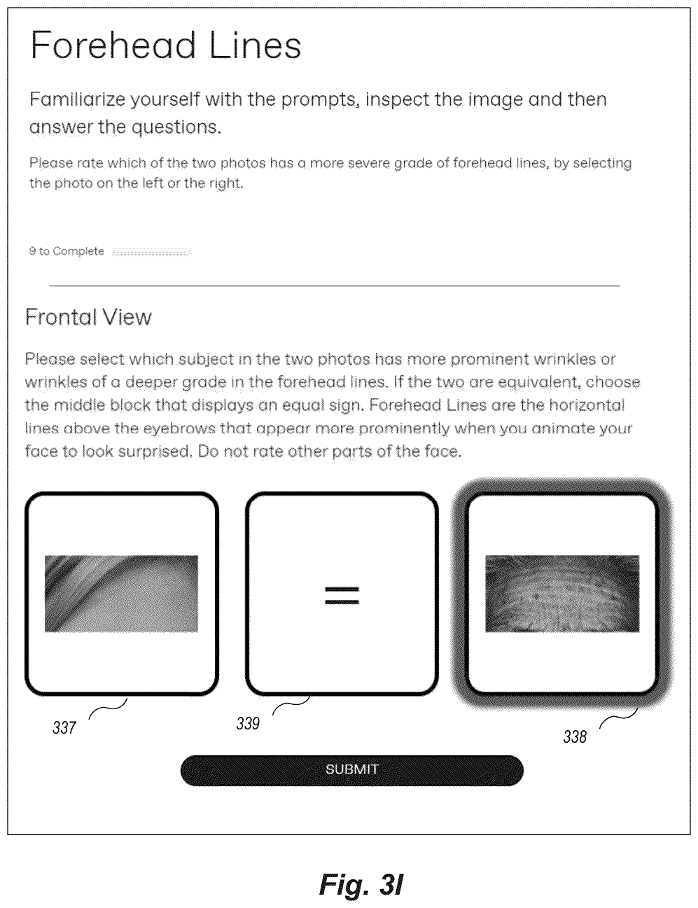

FIGS. 3A-3I are example screen displays and other illustrations that show a web based portal, an aesthetic visualization portal, for interacting with Aesthetic Delta Measurement System to generate crowd sourced labeled visual image data and to generate physician assisted ground truth data.

FIG. 3A illustrates a portion of the web based aesthetic visualization portal after successful login. This is the physician portal, which is currently separate from the crowd-based labeling platform portal. In other example ADMS interfaces, these access interfaces may be combined or arranged differently.

FIG. 3B is a screen display showing the different scales or characteristics/features 305 that the ADMS is interested in evaluating. The first scale of interest measures facial aging. The second scale of interest is used to measure neck volume. In overview, the user evaluates a series of images (photos or videos) to complete a survey. As shown in FIG. 3C, the first time the survey is executed, after a user consents to legal requirements, the aesthetic visualization portal (AVP) provides a set of instructions for how to complete the survey (e.g., scoring of the images). In particular, the severity or scale of facial aging is represented by an evaluation guide 306 having respective scalar values 307. A user is instructed to move slider 308 along scale 307 (corresponding to guide 306) to indicate where the user evaluates an image 309 (shown as a placeholder) along the scale 307. Each scalar position (which is a number or other indicator such as a color or other discrete value) of scale 307 corresponds to an guide image in guide 306. Any form of facial scale may be incorporated.

The user can evaluate the image 309 using three different facial views: frontal, oblique and lateral views (not shown). In each view, the user can place the image at a scalar position using the slider 308 as illustrated. FIG. 3C shows the slider 308 method applied to a frontal view shown in image 309. The user can drag the slider 308 left and right to adjust where the user wants to “place” the image 309 on scale 307. Placement of the image 309 on the scale 307 assigns the corresponding scalar value to the image (data structure that corresponds to the image). FIG. 3D illustrates a result of a user moving the slider 308 further to the right to a scalar position 311 (having a rating of 7.78) to indicate more facial aging severity is present in the image 309.

FIG. 3E illustrates guides and corresponding scales for an oblique view 312 and a lateral view 313. These icons are placeholders for icons/thumbnails that are more representative of oblique and lateral views. The user interface works as described relative to the frontal view to assign objective values to image 309 (not shown).

FIG. 3F is an example screen display for an interface for assigning objective measures of neck volume to an image. The interface for assigning such values to a frontal view of an person in image 315 along scale 322 according to guide 321 using slider 323 operates similarly to that described with respect to FIGS. 3C-3E. Instructions 320 are given first. The interface, in response to the user using slider 323 to move image 315 along scale 322, assigns the corresponding scalar value to the image. Of note scale 322 and guide 321 have only 5 major positions. In addition, there are only a frontal view and a lateral (side) view for assignment of neck volume values. Depending upon the aesthetic being measured, certain views may make more sense than others. In addition, different scales and guides may present different numbers of primary differentiation positions. Further, as shown in FIGS. 3A-3F photo-numeric scores are used (1-9, 1-5, etc.) Other ADMS examples may use different scoring or measurements.

FIGS. 3G-3I illustrate another portion of the web. In this interface, a different approach to assigning objective measurable criteria to images is used and the scoring is performed by the back end, the ADMS, to rank each images according to an entire population of images. Here, measurements are collected by presenting a series of image pairs to be compared (pairwise) only relative to each other, as one having more of the surveyed feature than the other, or as equal to the other. In this manner, the results of each pairwise comparison are used to dynamically rank the current image being labeled in the entire population of labeled images.

In particular, FIG. 3G is a screen display showing the characteristics/features the ADMS wants the “crowd” to use to evaluate and provide objective scoring for a plurality of images. The first feature of interest measures forehead lines 331. The second feature of interest is used to measure glabellar lines (glabellar lines are the vertical lines between the eyebrows and above the nasal bone that are positioned over the glabellar bone). In some versions of the crowd portal, the URL displayed to the user determines which measurements (which aesthetic feature review) is desired. For example, a different URL may point to a glabellar line image review than the URL used to access a forehead line image review.

As shown in FIG. 3H, the first time the survey is executed by a crowd participant, after the participant consents to legal requirements, the aesthetic visualization portal (AVP) provides a set of instructions 333 for how to complete the survey (e.g., scoring of the images). In particular, for each relevant view, for example the frontal view, the participant is asked to select which photo image of a pair of images, e. g., image 334 and image 335, contains more prominent wrinkles or wrinkles of a deeper grade in the glabellar lines. If the participant believes the images depict wrinkles in the glabellar lines that are equivalent, the participant is to choose block 336 to indicate equality. In the interface shown, the image/block selected is highlighted (here shown in red).

FIG. 3I illustrates a screen display of a similar interface to FIG. 3H for forehead lines. Here, for each relevant view, for example the frontal view, the participant is asked to select which photo image of a pair of images, e, g., image 337 and image 338, contains more prominent wrinkles or wrinkles of a deeper grade in the forehead lines. The forehead lines are the horizontal lines above the eyebrows. If the participant believes the images depict wrinkles in the forehead lines that are equivalent, the participant is to choose block 339 to indicate equality. In the interface shown, the image/block selected is highlighted (here shown in red).

Once a selection is made relative to a pair of images (in either the forehead or glabellar line analysis), the corresponding information regarding the selection is sent to the ADMS server 101 (or ADMS ranking service, not shown) where the ranking is computed (this may be also performed by other software ranking servers or services distributed throughout the ADMS environment).

In one example embodiment, the images (photos) to be analyzed pairwise are divided and put into bins of presumably similar images. For example, a set of 1000 images may be divided into 5 bins where a scale of 1-5 scalar values are at play. Then, a participant is tasked to compare a set of images from the same bin, in an effort to allow the system to rank all of the images within the bin using an ELO style rating system. ELO rating systems are typically used to stack rank players in zero-sum two-player games, when not all players can play all other players in the system (such as in chess). Other rating systems can be used by the ADMS ranking service. In one example ADMS, each participant is requested to analyze in a pairwise fashion a batch of photos comprising: 5 photos (images) against another single photo in a same single bin and then 1 photo in each of the other bins (9 total analysis comparisons in each survey).

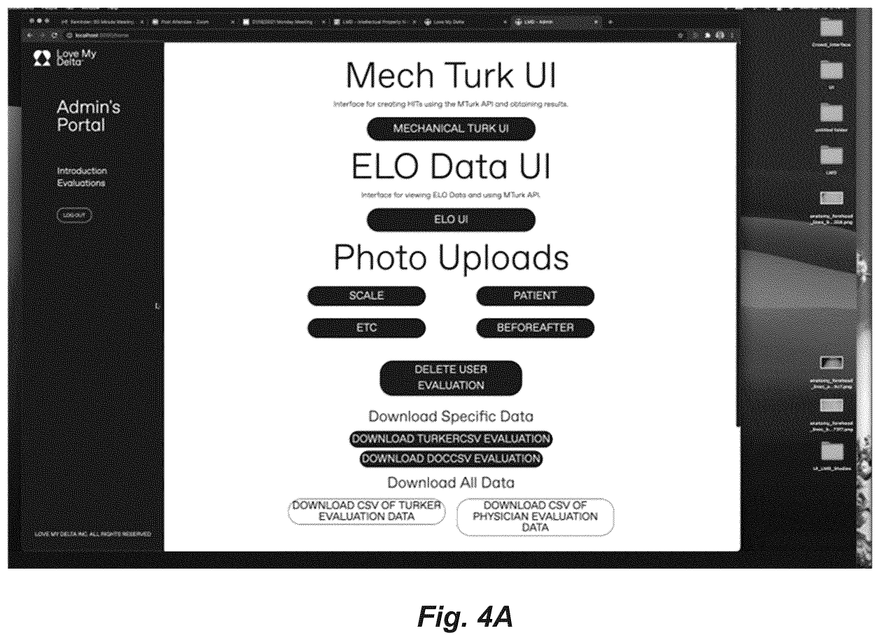

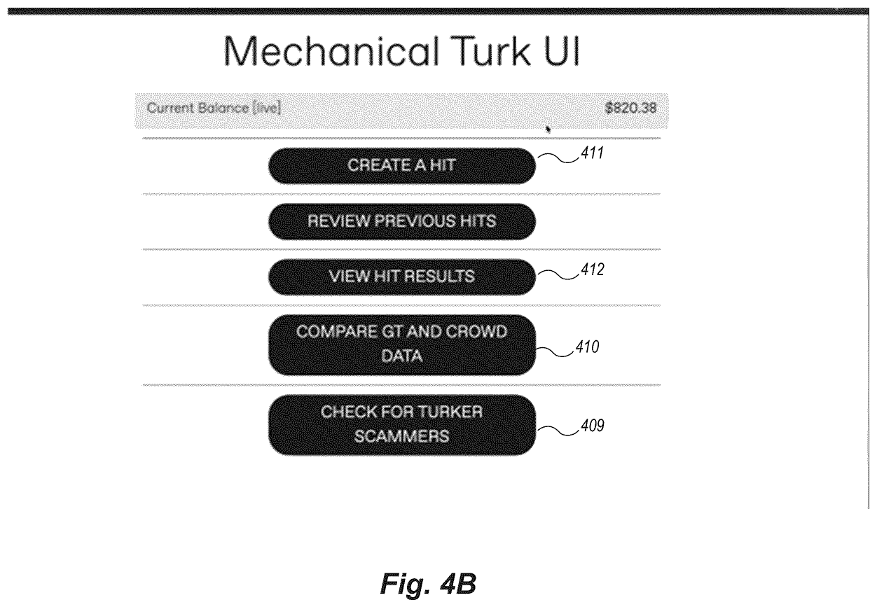

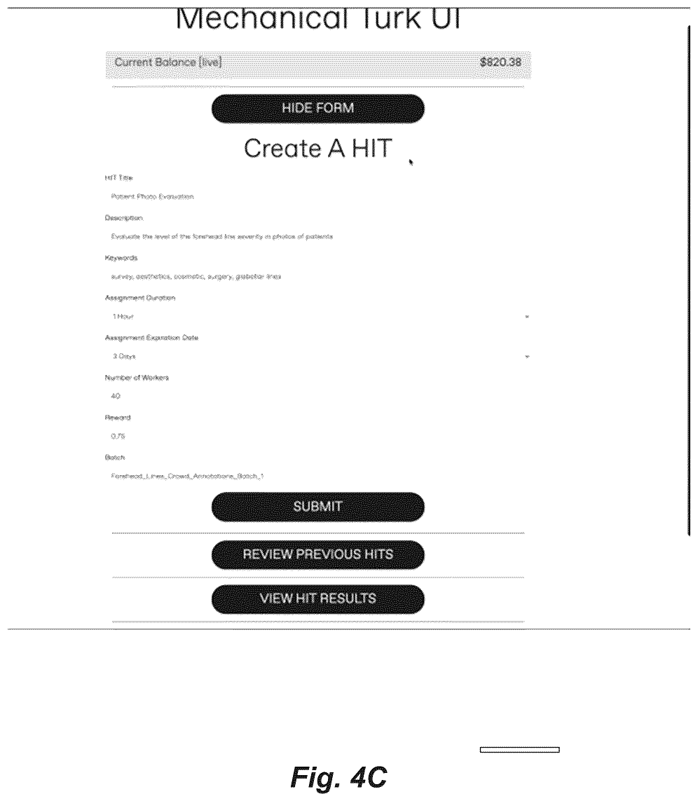

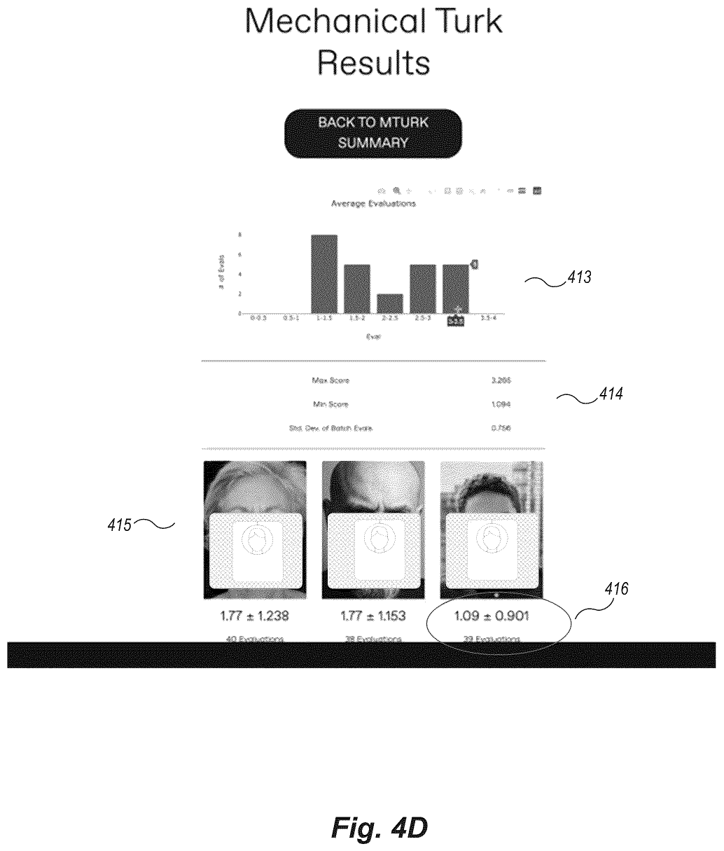

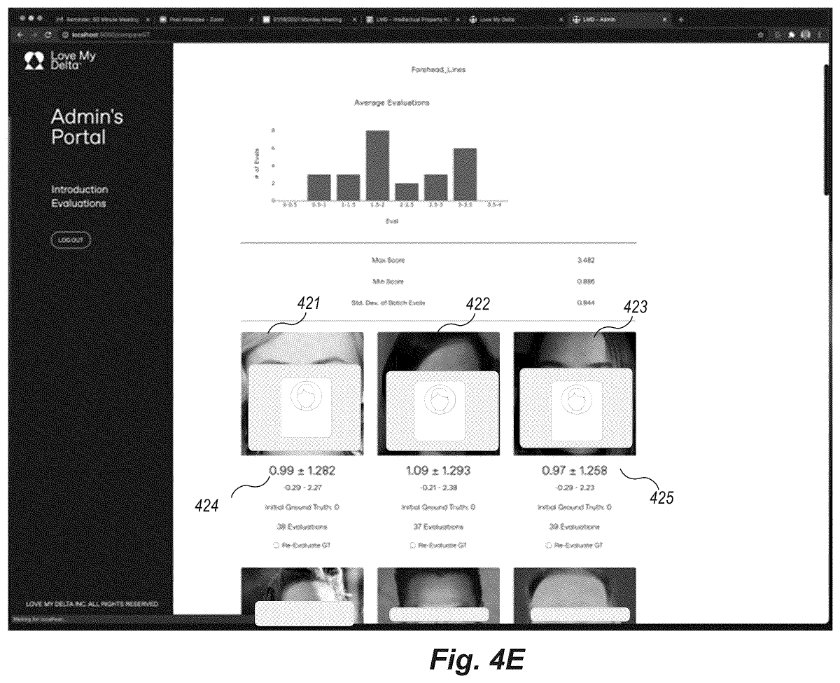

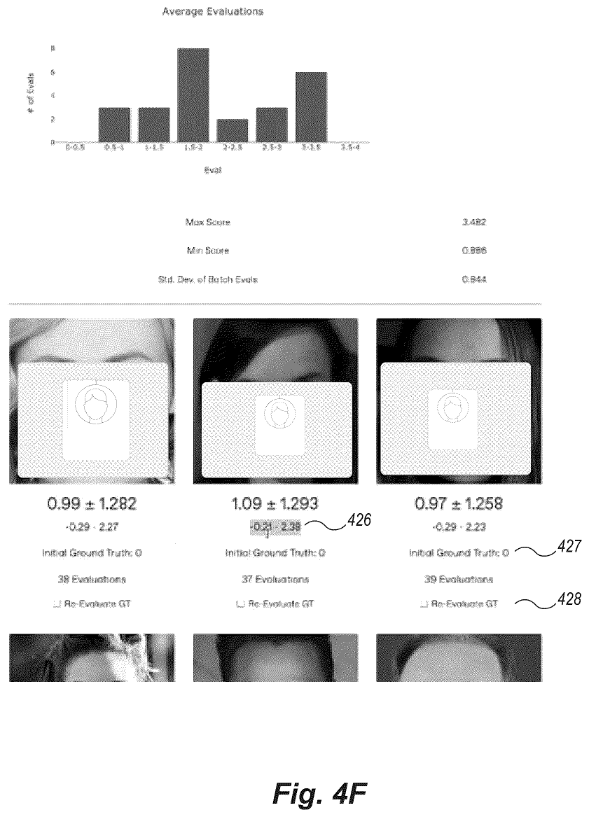

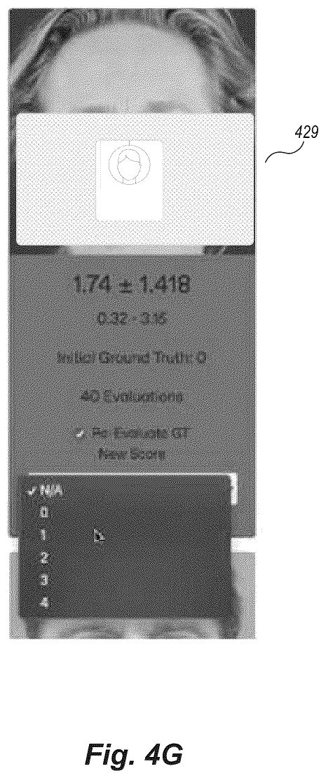

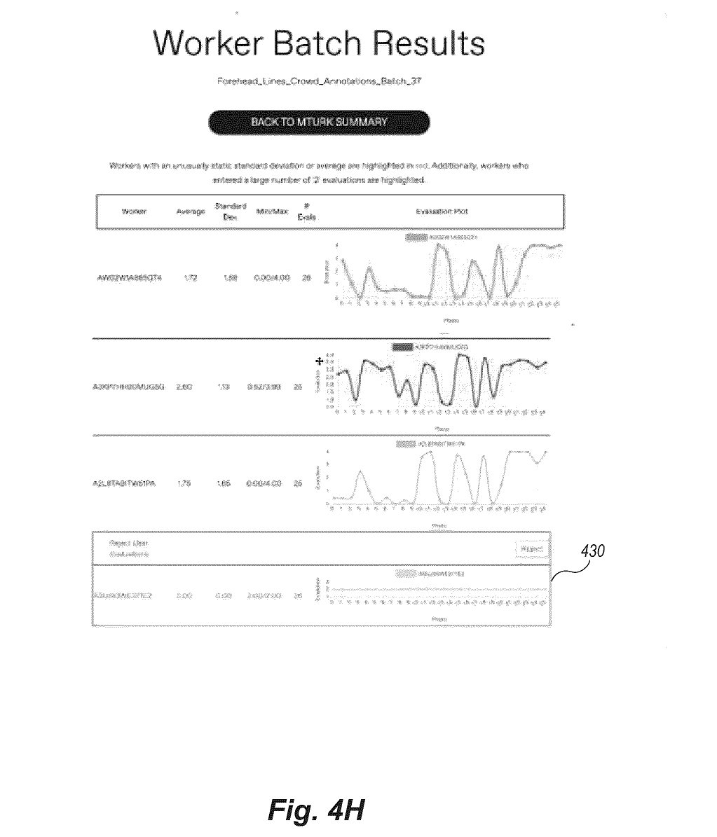

FIGS. 4A-4J are example screen displays that illustrate an administration portal to the web based aesthetic visualization portal used for crowd sourced data in an example Aesthetic Delta Measurement System. FIG. 4A illustrates the various functions available from the administrative view of the ADMS web portal. This view is used to manage Mechanical Turk engine support for administering and collecting data from the participant surveys, to provide an interface for visualizing the resultant ELO rankings, a console for managing, uploading and administering the photo images, and other administrative functions. FIG. 4B is a screen display illustrating the various functions available for controlling the Mechanical Turk (MT) engine through the MT API. In the first function 411, “create a HIT,” a crowd job is defined and initiated. The notion of a “HIT” is a survey assignment (Human Intelligence Task) for sending out “batches” of images to the crowd (a crowd worker job), which can be preconfigured. FIG. 4C is a detail screen display showing components of a hit when it is created and details the assignment duration, reward, number of works, description, when it expires, etc.

FIG. 4D is a detail screen display showing the results of selecting a batch from a screen (not shown), when the “view hit results” function 412 is selected in FIG. 4B. The results shown in FIG. 4D show the distribution of the average scores that each photo in the batch 413 received as well as the maximum minimum and standard deviation of these ratings. Further, the administrator can view the photos of each person in photo area 415 as well as the number of evaluations they received, and the average and standard deviation of their scores. For example, the score 416 indicates an average value of 1.09, a standard deviation of +/−.901, and 39 evaluations performed. These results give a visual representation of the objective scoring performed using crowd evaluated data.

FIG. 4E is a detail screen display showing the results of selecting a batch from a screen (not shown), when the “compare GT and crowd data” function 410 is selected in FIG. 4B on a particular batch of captured data. A batch is selected (not shown) for comparison. This function enables a comparison of manually input ground truth data and data resulting from crowd performed evaluations. A portion of the resultant comparison display is shown in FIG. 4F. In FIG. 4F, the interface shows similar information to that shown in FIG. 4D. Of particular interest for comparisons of crowd evaluated data to ground truth data is metric 426 which represents the average value minus the standard deviation value to the average value plus the standard deviation value (full range). If the ground truth (manually supplied data) is outside of this broader range (doesn't fall within the crowd evaluated range), then the image is flagged and the administrator is given the opportunity to easily reevaluate and reassign a new ground truth value. This is demonstrated in FIG. 4G which shows flagged image 429 and an interface for selecting a new score.

FIG. 4H is a screen display showing the results when the “check for Turker scammers” function 409 is selected in FIG. 4B. In essence, the administrator portal support checks each worker's average rating, their maximum and minimum rating, and displays a chart which shows their ratings for each photo. The interface automatically flags the worker, if their standard deviations of ratings is so small, that they're basically just repeating the same thing every single time. Chart 430 illustrates a worker who appears to be fraudulently misusing the system. As shown in a close up snapshot of the display screen in FIG. 4I, this worker appears to be scoring all photos in his/her batch with the same number—a 2. The administrative is then given an opportunity to remove such results (using the MT API), thereby preventing any skew of otherwise objective results.

FIG. 4J is a screen display illustrating a visualization and use of the ELO ranking data being accumulated. In FIG. 4J, a bar chart visualization 443 of all of the ELO rankings for forehead line scores 440 is shown. The buckets/bins are differentiated by the red lines in graph 443. The number of photos that differ from the original ground truth data are shown in list 441 relative to the number of classes they differed (list 442) from their corresponding ground truth data. For example, this table shows that only 27 photos were “off” from their respective ground truth data by 2 classes (scores), whereas 399 photos were “off” by 1 class. Such analysis provides an effective check on ground truth data when such data is being curated and not already known. For example, if some type of visual objective measurement of images (for whatever purpose) is to be analyzed, and the characteristics for such measurement and evaluation aren't clear, and little ground truth data exists or is agreed on, the techniques presented here may be used to determine an initial set of ground truth data. This analysis also illustrates the power of combining manually curated ground truth data with crowd sourced data to improve accuracy and reliability of a machine learning system. Upon selection of the “review forehead ELO scores” user interface control 445, the administrator can review each of the individual images (close ups) in each scalar bin for further review (not shown).

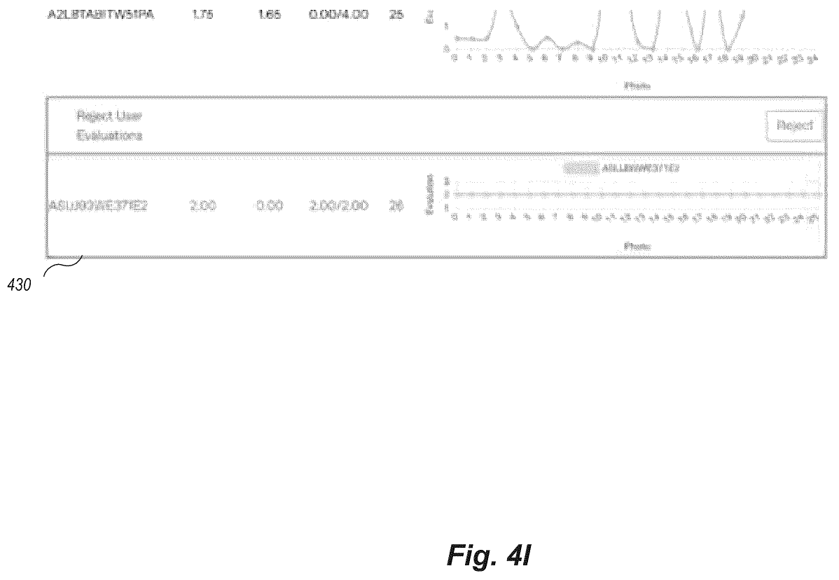

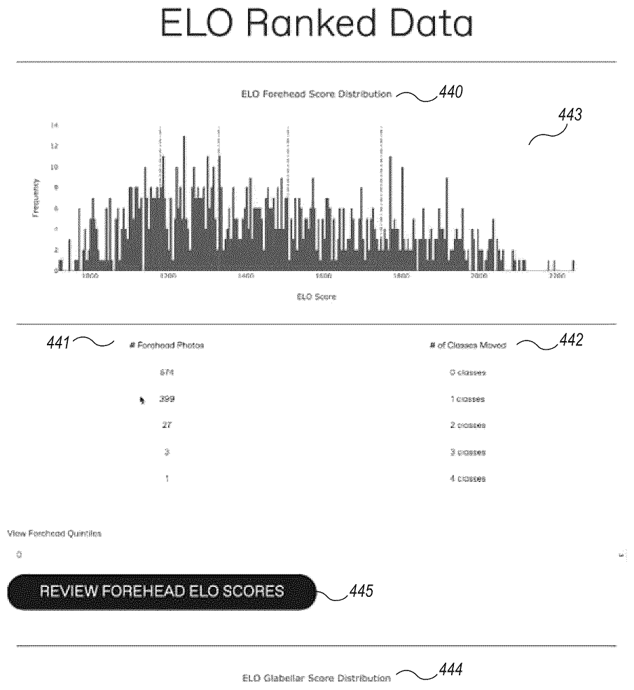

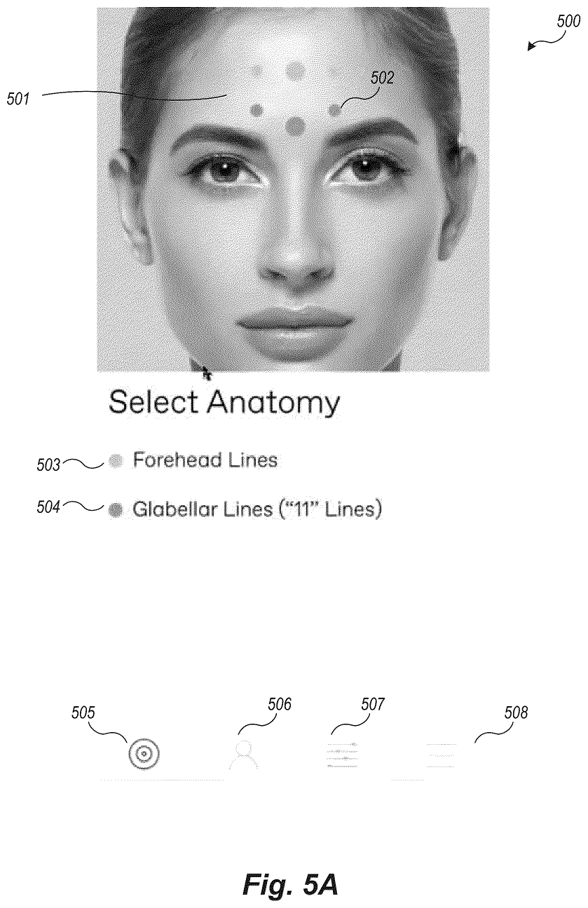

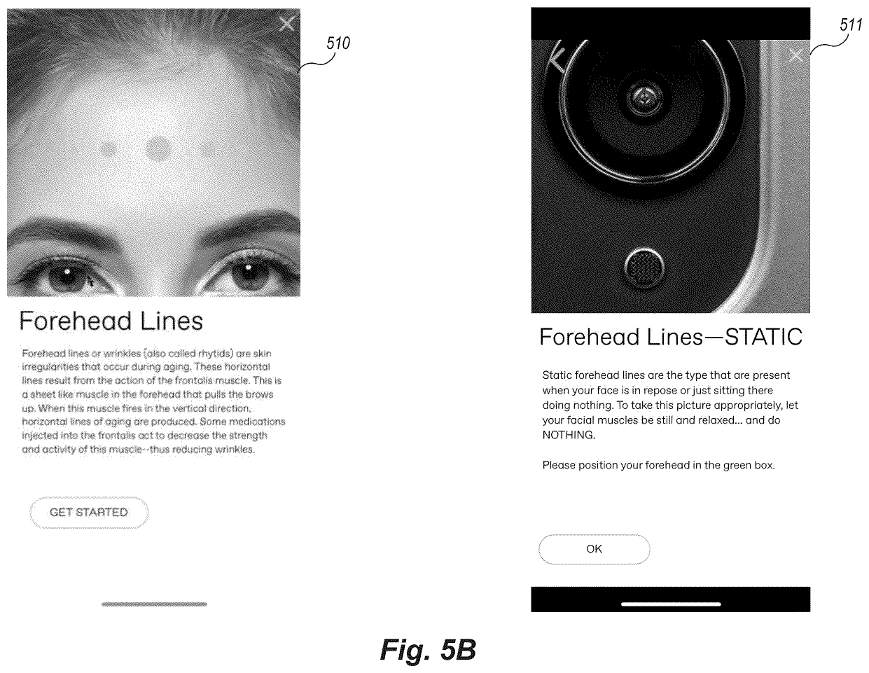

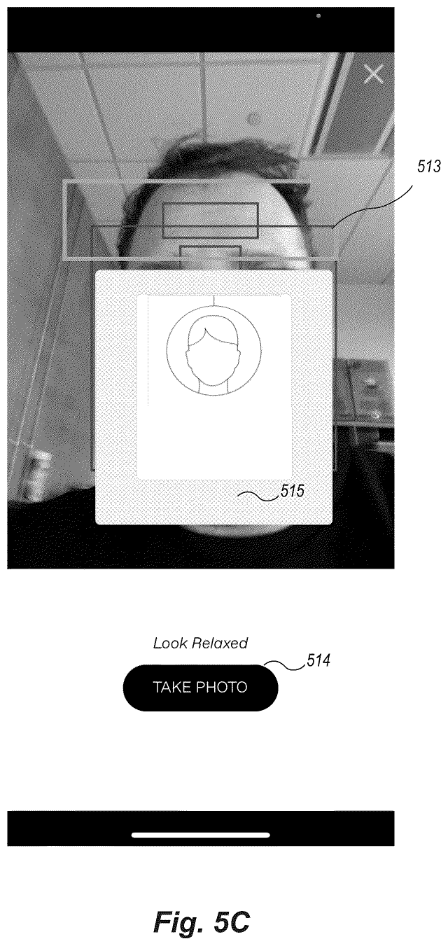

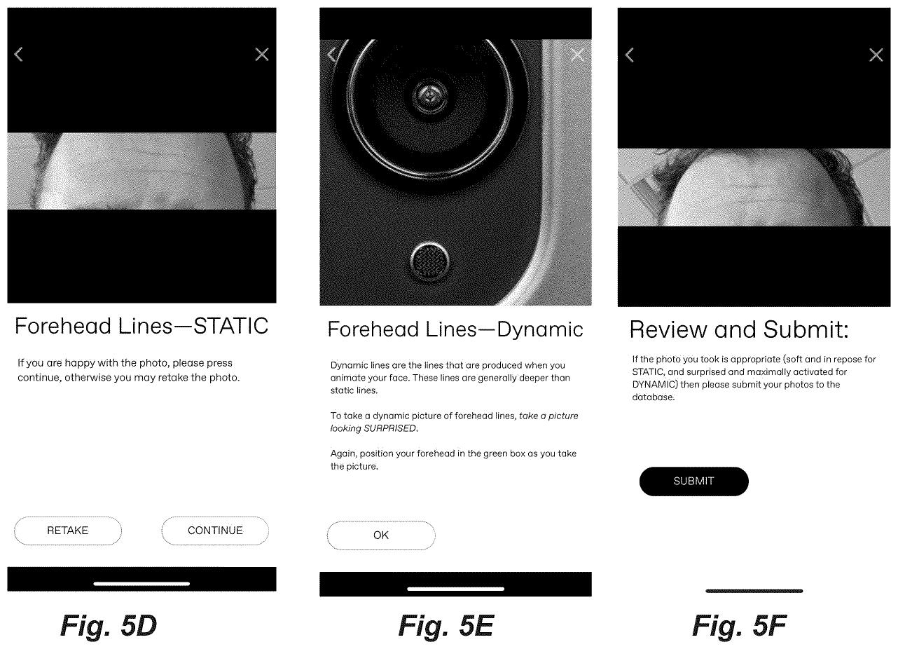

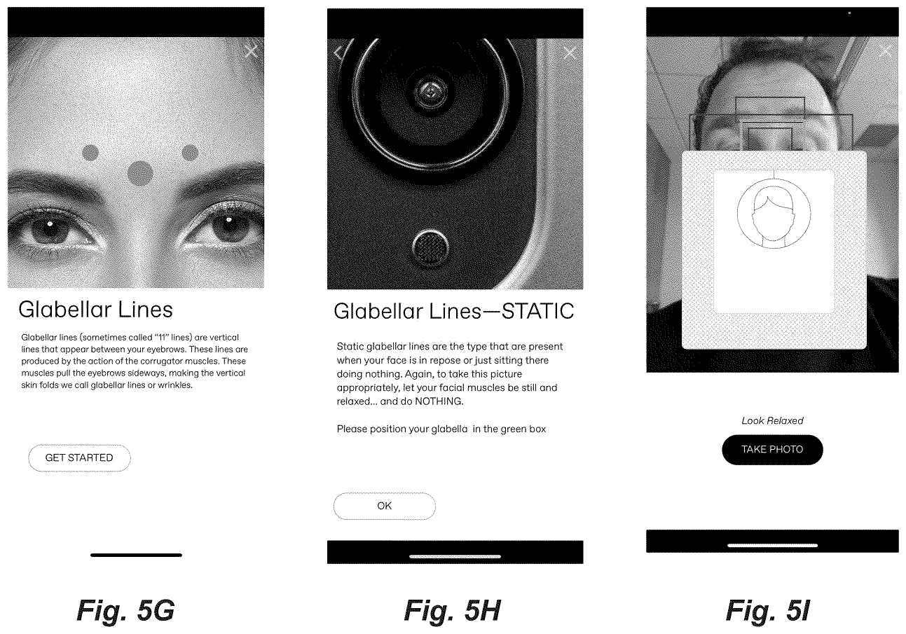

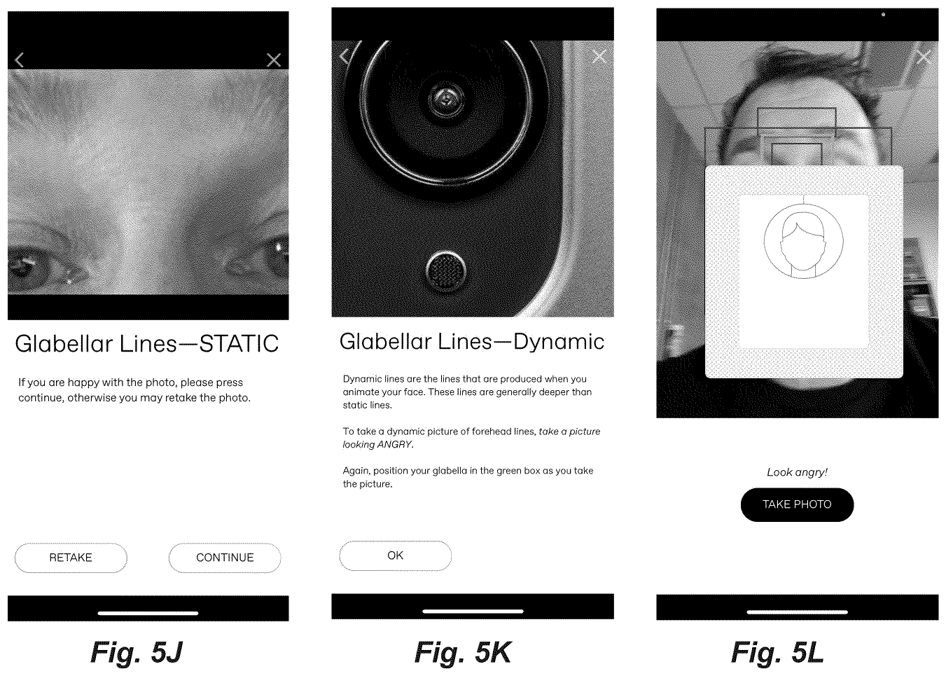

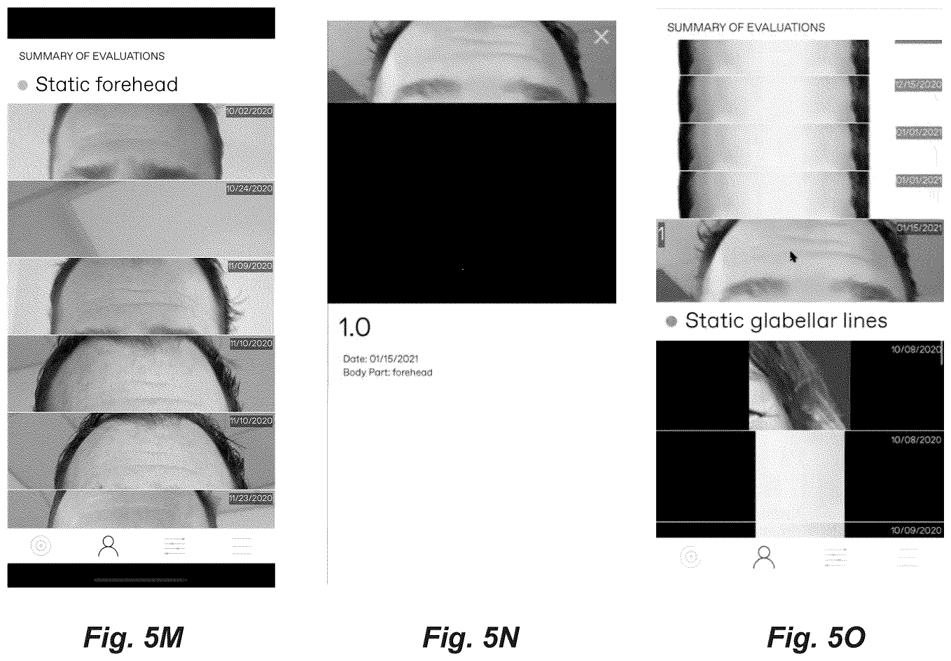

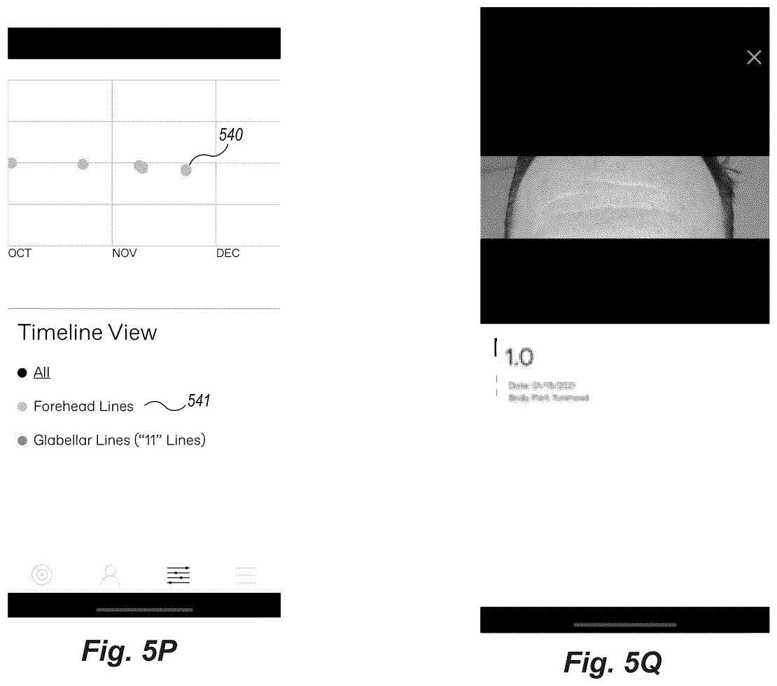

FIGS. 5A-5Q are screen displays from an example Aesthetic Delta Measurement System illustrative of a personal analysis application for viewing an individual's aesthetic changes over time. This application corresponds to phone application (consumer app) 202 in FIG. 2. Phone user interface 202 is directed to providing analysis tools for information regarding an individual's aesthetic features currently and over time. It also provides another manner for obtaining objective labeling of aesthetic images which are forwarded to the ADMS server 101 for machine learning purposes.

After logging in and agreeing to any presented terms of use, privacy etc., the user is greeted by the main display for navigating the application as shown in FIG. 5A. In FIG. 5A, the main display 500 shows which area of the image (in this case facial features), the user is interested in analyzing. For example, facial region 501 comprises two different types of dots (here in different colors) to illustrate that the user can choose to work with forehead lines or glabellar lines. The user can either select a dot (such as dot 502) or one of the appropriate links 503 and 504 to navigate to the appropriate collection screens.

The main display 500 offers four navigation user interface (UI) controls 505-508 at the bottom. The first UI control (target) 505 is for capturing images of the individual operating the phone. The second “people” UI control 506 is for navigating to a history of images taken for that individual, meant to show differences (the delta) of an aesthetic view over time. The abacus UI control 507 is for navigating to a timeline of images, again to show differences over time, and allows various filtering control. The “hamburger” menu UI control 508 is for access to other information such as privacy policy, terms of use, etc.

FIG. 5B shows the results of user selection of forehead line capture and analysis, for example by selecting a yellow dot in region 501 or by selecting forehead line link 503. As shown, the user is navigated through a set of instruction screens 510 and 511 regarding the type of aesthetic capture to be performed. Although not reflected in the text, the user is prompted in the next screens to perform a capture within a bounding rectangle, shown as green rectangle 513 in FIG. 5C. The green rectangle signifies where the user is supposed to frame his/her forehead in the image. The user is instructed to take a photo by pressing UI control (button) 514. (To protect confidentiality, the actual faces of persons in many of the photos described herein are obfuscated by an object such as object 515 in the Figures throughout this description.) In some example ADMS phone applications, when the user fails to situate his/her head properly within the bounding rectangle, the user interface changes in some way, such as by making the color of the bounding rectangle less vivid or by changing the color to yellow instead of green. Other feedback mechanisms such as audio or haptic feedback are also possible. The blue bounding rectangles also presented in FIG. 5C are debugging rectangles which are used to calculate the location of the forehead and the glabella region using internal feature and image recognition software present in the phone itself. Other visual prompts (not shown) may also be present. In FIG. 5D the user is prompted to retake or accept the photo in display 516.

In some example ADMS application, photos taken statically (no intentional movement of the forehead/glabellar lines) are taken as well as dynamically. FIG. 5E illustrates a screen display for instructions on taking dynamic photos of forehead lines. Similar to the process of capturing static forehead lines, FIG. 5F illustrates a screen display for reviewing and submitting a captured image of forehead lines but resulting from movement. The forehead lines captured as a result of movement in FIG. 5F are more pronounced that those captured statically, FIG. 5D.

In one example ADMS, these images are forwarded to the ADMS server 101 and data storage 102 in FIG. 1 to be scored using machine learning engines/models as described above In some ADMS examples, the scores are presented to the user; in some examples they are used to enhance machine learning datasets, for example to acquire more training, test, and validation data.

FIGS. 5G-5L are screen displays used for glabellar line capture and works similarly to capture of forehead lines described with reference to FIGS. 5B-5F. These screen displays result from an individual selecting the glabellar line link 504 (FIG. 5A) to navigate to glabellar line capture. The sequence shown in FIGS. 5G-5L operate in the same manner as that described with reference to FIGS. 5B-5F including both static and dynamic capture. As the glabellar area is in a different position on the human face, the bounding rectangle shown in FIGS. 51 and 5L is appropriately relocated and redraw by the capture software. The confirmation and submit screens (e.g., for scoring or retaking the photos) are not shown.

FIGS. 5M-5O are screen displays resulting from user selection of the people UI control 506 in FIG. 5A to navigate to an image history showing evaluations of aesthetic features captured for that individual over time. For example, FIG. 5M shows a series of images of forehead static captures annotated with a label regarding when they were captured. Similar images are displayed (not shown) for the other captures taken dynamically and for glabellar lines, e.g., as displayed in FIG. 5O. A particular image may be selected, and in some example ADMS applications, for example as shown in FIG. 5W, the score and other metadata and/or annotations may be displayed (e.g., body part, date, score, and other data, such as treatment data. Using such a history, the individual can observe objective measurements of his/her aesthetic history, for example as a result of a series of aesthetic wellness and health treatments over time.

FIGS. 5P-5Q are screen displays resulting from user selection of the people abacus (timeline) UI control 507 in FIG. 5A to navigate to a timeline visual of objective scores of the aesthetic features captured for that individual over time. For example, in FIG. 5P, each “dot” 540 (or other indicator) corresponds to a photo and is associated with an objective measurement determined by the ADMS server (e.g., ADMS server 101 in FIG. 1). As well, in some example interfaces, a score displayed as in FIG. 5P may be different or modified from that of the ADMS server, for example so as to convey more or different information. As displayed, the different types of captures (corresponding to different aesthetic features such as forehead lines or glabellar lines) can be indicated using different visuals, such as different colored dots. As well, the display can be filtered to only shown certain designated types of aesthetic features (e.g., by selecting filtering link 541). An individual can also navigate to the scoring of a particular image by selecting one of the dots, such as dot 540, which results in FIG. 5Q, similar to FIG. 5N.

In addition to the interface shown, some personal analysis applications for an example ADMS also includes an ability for the individual to contribute to the ADMS aesthetic data repository. This device annotation capability operates similarly to the interface described for crowd workers/participants in the web-based aesthetic visualization portal used for crowd sourced data described with reference to FIGS. 3G-3I. In this scenario, the individual would sign up and agree to answer “surveys” regarding aesthetic appearance data of other persons. The interface can operate using the pairwise comparisons described with reference to FIGS. 3G-3I or the guide/scale indications described with reference to FIGS. 3A-3F. In either case, the obtained evaluation and objective measurement data is forwarded to the ADMS and associated data repository for further use in machine learning and analysis. Other annotations and labeling could also be performed.

### Facial Feature Recognition and Patch Determination

As described above with reference to FIGS. 5A-5Q, in order to provide the acquisition of images on the consumer (phone) application, the ADMS provides bounding rectangles allow the user to position the phone camera correctly to acquire data.

The application uses facial landmark detection algorithms to help the user position their picture correctly and to determine the regions of interest (patches) within the image that should be analyzed by the remainder of the machine learning pipeline. For example, different bounding rectangles are provided for measuring glabellar lines versus forehead lines (compare FIG. 5C with FIG. 51).

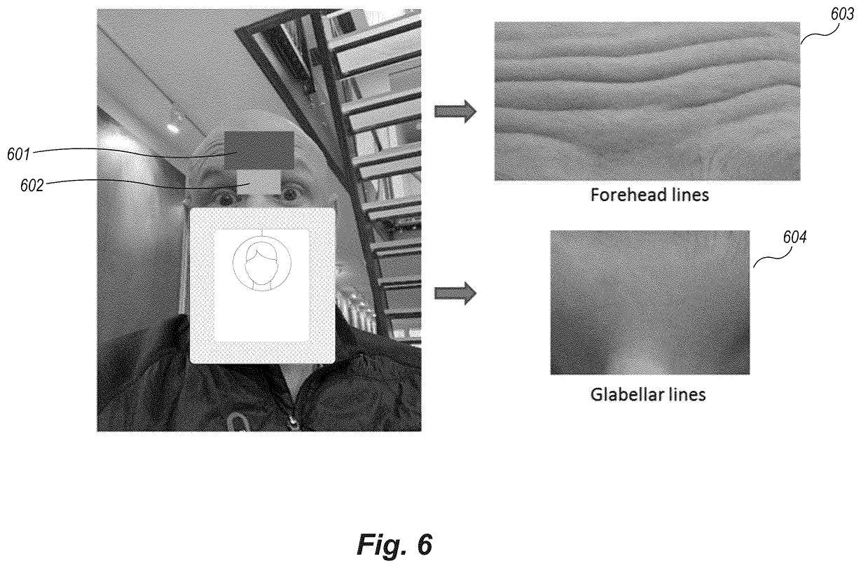

The facial landmarks are extracted in near real-time as the user is holding the camera and bounding boxes around the detected regions of interest are shown to help with the positioning. FIG. 6 illustrates the different bounding rectangles 601 and 602 that are provided for acquisition of forehead lines 603 and glabellar lines 604, respectively. Once satisfied the user can take the picture or it can be taken automatically depending upon the configuration.

In an example ADMS, the pupils of the individual are the facial landmark used to determine a correct location of the forehead region and glabellar region. Other landmarks may be similarly incorporated. Currently, facial landmark detection is based on keypoint detection in images. A traditional architecture is stacked hourglass networks, but other approaches have emerged in recent years and are frequently published. The ADMS is operative with any and all of these approaches. For very common landmarks (such as pupils) off-the-shelf detectors can be used (e.g., the facial feature detection included in the iOS SDK on iOS devices).

As aesthetic feature acquisition is extended from simple frontal facial areas of interest to lateral face views and body imagery, landmark detection is similarly customized and adapted to one or more of keypoint detection algorithms used in pose estimation for this purpose (e.g., stacked hourglass networks).

The application then forwards (e.g., sends, communicates, etc.) the extracted images to a server side application (such as running on the ADMS 101 in FIG. 1) for further processing.

### Machine Learning Environment and Considerations

Example ADMS environments use traditional machine learning pipelines. Once data is acquired as just described, the next step in the machine learning pipeline is to take the extracted regions (the determined patches) from step 1 and rate them on an appropriate scale (see FIGS. 3A-3F) or rank them as described further below using an ELO ranking algorithm (see also FIGS. 3G-3I).

The rating/scaling is used to generate the training data set for the ADMS machine learning based rating algorithm. The machine learning models take into account both trusted human data (just as seed data) and data from both untrusted crowd data interfaces and data from user applications associated with aesthetic procedures who can view their own changes over time. In some ADMS environments, the ADMS may guide the human labeling process so that annotators are steered towards areas where its models are underperforming. For example, suppose that the data in one geolocation of the world includes a different population composition than a second geolocation (for example, the latter might have a younger population of or different ethnicities). This results in an active learning feedback loop which ultimately enhances the precision and recall of the predictive ratings.

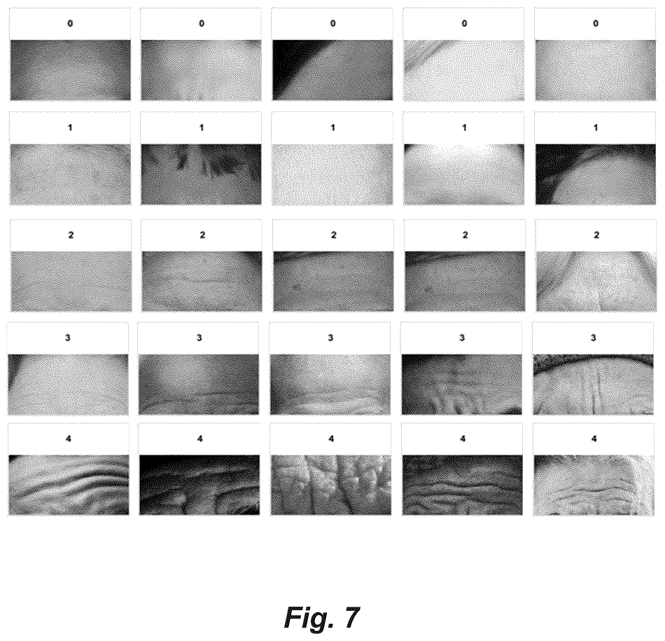

One example ADMS machine learning environment uses convolutional neural networks (CNN) typically used in the context of image classification to assign each image a position on a scale. The CNN is trained based on the manually (crowd or individual) ranked training using CNN architectures such as VGG16, ResNet-50 and other variations) either as a classification (assign image to discrete/scalar categories, e.g., 0, 1, 2, 3, 4, 5) or a regression model (predict “soft” ELO score directly, e.g., score between 800 and 1600). FIG. 7 illustrates an example ranking of forehead lines on a discrete scale as produced by the trained (classification) model.

The choice of CNN architecture is made based on performance on the given dataset (aesthetic area of interest) and may vary from model to model. Ultimately CNNs are particularly well suited to this task due to their outstanding performance in image classification.

### Image Scaling and Image Ranking Procedure

As described with reference to the crowd interface of the web portal in FIGS. 3A-3F and the admin portion in FIGS. 4A-4C, one example ADMS uses the Mechanical Turk (MT) web platform for crowdsourcing. MT is used to employ some number (e.g., 40) of independent workers to navigate through groups of photos with an attached scale. These workers will give their opinion of the state of the subject's photo on a sliding scale with precision to the hundredth of a point. These data are then aggregated to create overall scores for a user's photo.

In a similar manner to ADMS' use of MT, in some example ADMS environments, users submitting new images through the web-based or app-based platform can be prompted to serve as a worker who rate photos through the platform. This feature can be useful to further train the machine learning models using “trusted” human data (e.g., the participant users of the systems that are receiving procedures) in addition to the untrusted crowd data obtainable through MT. This enables the machine learning environment to Users may opt to rate pairs of photos from other platform users in exchange for added features and functionality within the rating platform. The AMDS utilizes a weighted average of ratings obtained from MT (crowd sourced) with those from participant users of the platform as a part of the ranking service. This weighting can be configurable.

As described with reference to the crowd interface of the web portal in FIGS. 3G-3I, one example ADMS uses a ranking service (application, server, or the like) to dynamically recompute rankings for the entire population of images based upon a newly acquired image. One ADMS ranking service incorporates an ELO rating system to stack the images.

To perform an ELO scoring methodology (others can be used), A history of the value for K (a coefficient explained below) is stored in the database so that the K-coefficient can be reverse engineered and re-calculated if necessary. It is contemplated that the K-coefficient could be dynamically changed based on a number of variables that more accurately reflect a person's physical trait such as:
average amount of change in ELO score
number of competitions (comparisons) performed
quintile placement of patient before and after competitions as compared to the ADMS machine learning algorithm's initial “guess”
number of responses obtained for a specific competition of two photos Other variables could be similarly incorporate.

The logic for computing the ranking of a new image (image evaluation) is performed by the ADMS is as follows:
1) Identify Trait-Sex-Fitzpatrick strata of the new image
2) Use Neural Network algorithm to identify initial aesthetics rating quintile (Q1 through Q5)
3) Select a base set of n=5 existing images (x) within the quintile of the ML-based rating (*) of the new image. Also select n=1 randomly selected image (o) from each of the other quintiles of the Trait-Sex-Fitzpatrick strata. (see Figure) Total of 9 comparison images. Obtain 10 pairwise crowd-based ratings for the new image against the 9 comparison images. (10*9=90 total ratings)
4) Tabulate the number of ratings cast in favor of the new image against the number of ratings cast in favor of the comparison image.
   a. N_new>N_comparison->the new image is the winner
   b. N_new<N_comparison->the old image is the winner
   c. N_new=N_comparison->the images are tied.
5) Run Elo scoring analysis of the new image against the 9 other images. If the Elo score for the new image is:
   a. Bounded within the rating quintile, then record the Elo score for the new image and update Elo scores of comparison images.
   b. Outside of the Elo rating quintile, then randomly select 5 new comparison images from the appropriate rating quintile and reevaluate with 10 pairwise crowd-based ratings. Crowd-based ratings can be obtained using Amazon Mechanical Turk or through the LMD user-base (the phone application 120a-b of FIG. 1).

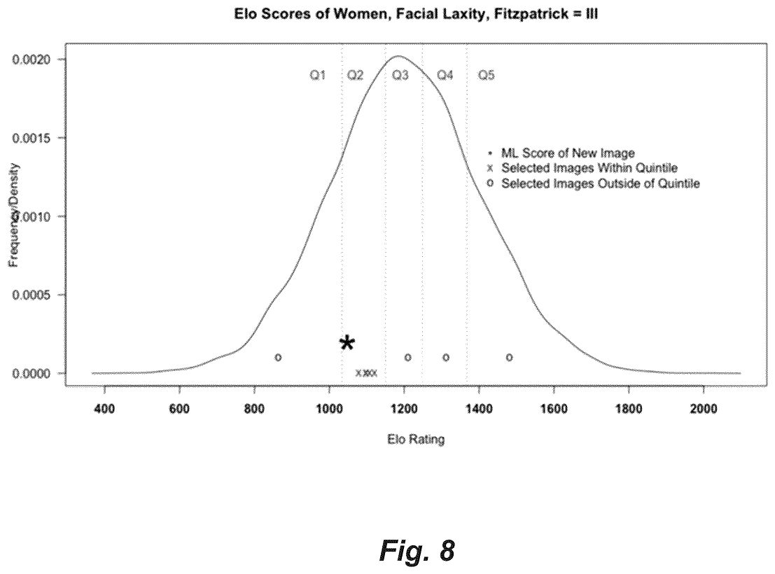

FIG. 8 is an example chart illustrating the quintiles and results used for ELO scoring of facial aging in women.

### Computing the ELO and ADMS Score

The Elo score for a new image (New) may be calculated by comparing to an existing image (Old′) after they are evaluated against one another. Given initial Elo scores:
ELO_new=initial score is ML predicted Elo score for a new image
ELO_old=current Elo score of existing image

Calculate the following quantities:
R_{new}=10^{(r}^{_{new}}^{/400)}
R_{old}=10^{(r}^{_{old}}^{/400)}
E_{new}=R_{new} [R_{new}+R_{old}]
E_{old}=R_{old}/[R_{new}+R_{old}]

where
S_{new}=1->if New image ‘wins’
S_{new}=0.5->if tied
S_{new}=0->if Old image ‘wins’
S_{old}=1->if Old image ‘wins’
S_{old}=0.5->if tied
S_{old}=0->if New image ‘wins’

Update new Elo scores as follows:
ELO_{new}′=ELO_{new}+K_{new}*[S_{new}−E_{new}]
   where K_{new} is bigger (K=40)
ELO_{old}′=ELO_{old}+K_{old}*[S_{old}−E_{old}]
   where K old is small (K=2−5)

The final ‘ADMS score’ for a given image is calculated as the percentile of the image's ELO score within that image's Trait-Sex-Fitzpatrick and 5-year age strata. The ADMS score within important user demographic strata will always be bounded between 0 and 100, where 0 indicates lowest rated image and 100 indicates the highest rated image. The 0 to 100 ADMS score is therefore comparable across images across body regions within the same individual user, as well as images obtained for a given user over time. Ratings obtained prior to appearance-enhancing treatments may be compared to post-treatment scores as the difference in ADMS scores, as well as percentage improvements. Very high and very low percentiles can be presented as ‘>99’ (for example) for any ADMS score.

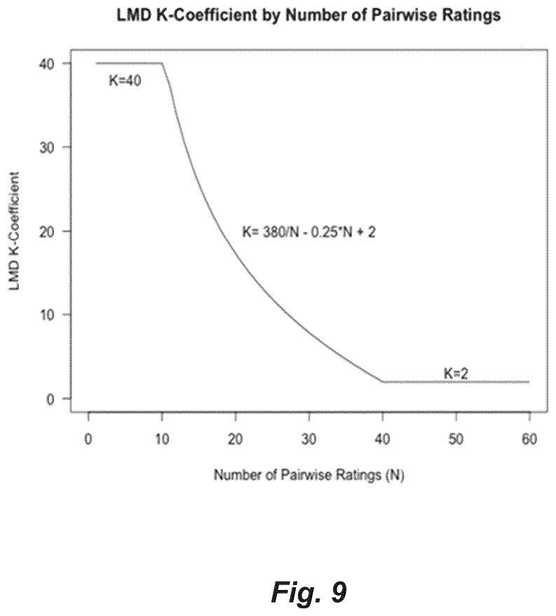

The ADMS can use a dynamic value of K that is tailored to each image and individual over time. New images can be subjected to higher values of K=40 to allow for greater levels of change in the ADMS score as the image is subjected to more comparisons. As the number of pairwise comparisons (N) increases from 10 ratings to, for example, 40 ratings, ADMS K-coefficient will gradually reduce to K=2 according to the power curve illustrated in FIG. 9.

### Example Computing System

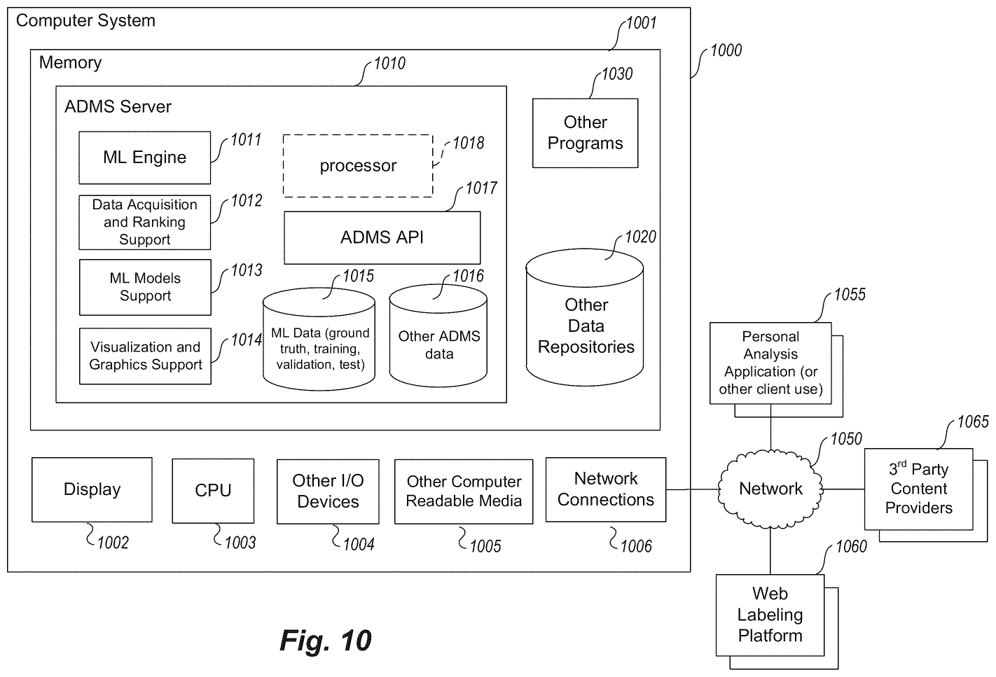

FIG. 10 is an example block diagram of a computing system for practicing embodiments of an example Aesthetic Delta Measurement System server including example components. Note that one or more general purpose virtual or physical computing systems suitably instructed or a special purpose computing system may be used to implement an ADMS server. Further, the ADMS server may be implemented in software, hardware, firmware, or in some combination to achieve the capabilities described herein.

Note that one or more general purpose or special purpose computing systems/devices may be used to implement the described techniques. However, just because it is possible to implement the Aesthetic Delta Measurement System on a general purpose computing system does not mean that the techniques themselves or the operations required to implement the techniques are conventional or well known.

The computing system 1000 may comprise one or more server and/or client computing systems and may span distributed locations. In addition, each block shown may represent one or more such blocks as appropriate to a specific embodiment or may be combined with other blocks. Moreover, the various blocks of the Aesthetic Delta Measurement System 1010 may physically reside on one or more machines, which use standard (e.g., TCP/IP) or proprietary interprocess communication mechanisms to communicate with each other.

In the embodiment shown, computer system 1000 comprises a computer memory (“memory”) 1001, a display 1002, one or more Central Processing Units (“CPU”) 1003, Input/Output devices 1004 (e.g., keyboard, mouse, CRT or LCD display, etc.), other computer-readable media 1005, and one or more network connections 1006. The ADSM Server 1010 is shown residing in memory 1001. In other embodiments, some portion of the contents, some of, or all of the components of the ADSM Server 1010 may be stored on and/or transmitted over the other computer-readable media 1005. The components of the Aesthetic Delta Measurement System 1010 preferably execute on one or more CPUs 1003 and manage the acquisition and objective measurement and evaluation use of aesthetic features and images, as described herein. Other code or programs 1030 and potentially other data repositories, such as data repository 1006, also reside in the memory 1001, and preferably execute on one or more CPUs 1003. Of note, one or more of the components in FIG. 10 may not be present in any specific implementation. For example, some embodiments embedded in other software may not provide means for user input or display.

In a typical embodiment, the ADSM Server 1010 includes one or more machine learning (ML) engines or models 1011, one or more data acquisition tools, ranking services and support 1012, one or more ML model support 1013 (for supporting the ML implementation, storage of models, testing and the like, and visualization and graphics support 1014. In addition, several data repositories may be present such as ML data 1015 and other ADMS data 1016. In at least some embodiments, some of the components may be provided external to the ADMS and is available, potentially, over one or more networks 1050. Other and/or different modules may be implemented. In addition, the ADMS may interact via a network 1050 with application or client code 1055 that, for example, acquires and causes images to be scored or that uses the scores and rankings computed by the data acquisition and ranking support 1012, one or more other client computing systems such as web labeling/annotating platform 1060, and/or one or more third-party information provider systems 1065, such as provides of scales/guides to be used in the visualizations and ML predictions. Also, of note, the ML Data data repository 1016 may be provided external to the ADMS as well, for example in a data repository accessible over one or more networks 1050.

In an example embodiment, components/modules of the ADSM Server 1010 are implemented using standard programming techniques. For example, the ADSM Server 1010 may be implemented as a “native” executable running on the CPU 103, along with one or more static or dynamic libraries. In other embodiments, the ADSM Server 1010 may be implemented as instructions processed by a virtual machine. A range of programming languages known in the art may be employed for implementing such example embodiments, including representative implementations of various programming language paradigms, including but not limited to, object-oriented, functional, procedural, scripting, and declarative.

The embodiments described above may also use well-known or proprietary, synchronous or asynchronous client-server computing techniques. Also, the various components may be implemented using more monolithic programming techniques, for example, as an executable running on a single CPU computer system, or alternatively decomposed using a variety of structuring techniques known in the art, including but not limited to, multiprogramming, multithreading, client-server, or peer-to-peer, running on one or more computer systems each having one or more CPUs. Some embodiments may execute concurrently and asynchronously and communicate using message passing techniques. Equivalent synchronous embodiments are also supported.

In addition, programming interfaces to the data stored as part of the ADMS server 1010 (e.g., in the data repositories 1016 and 1017) can be available by standard mechanisms such as through C, C++, C#, and Java APIs; libraries for accessing files, databases, or other data repositories; through scripting languages such as XML; or through Web servers, FTP servers, or other types of servers providing access to stored data. The repositories 1016 and 1017 may be implemented as one or more database systems, file systems, or any other technique for storing such information, or any combination of the above, including implementations using distributed computing techniques.

Also the example ADMS server 1010 may be implemented in a distributed environment comprising multiple, even heterogeneous, computer systems and networks. Different configurations and locations of programs and data are contemplated for use with techniques of described herein. In addition, the [server and/or client] may be physical or virtual computing systems and may reside on the same physical system. Also, one or more of the modules may themselves be distributed, pooled or otherwise grouped, such as for load balancing, reliability or security reasons. A variety of distributed computing techniques are appropriate for implementing the components of the illustrated embodiments in a distributed manner including but not limited to TCP/IP sockets, RPC, RMI, HTTP, Web Services (XML-RPC, JAX-RPC, SOAP, etc.) and the like. Other variations are possible. Also, other functionality could be provided by each component/module, or existing functionality could be distributed amongst the components/modules in different ways, yet still achieve the functions of an ADMS.

Furthermore, in some embodiments, some or all of the components of the ADMS Server 1010 may be implemented or provided in other manners, such as at least partially in firmware and/or hardware, including, but not limited to one or more application-specific integrated circuits (ASICs), standard integrated circuits, controllers executing appropriate instructions, and including microcontrollers and/or embedded controllers, field-programmable gate arrays (FPGAs), complex programmable logic devices (CPLDs), and the like. Some or all of the system components and/or data structures may also be stored as contents (e.g., as executable or other machine-readable software instructions or structured data) on a computer-readable medium (e.g., a hard disk; memory; network; other computer-readable medium; or other portable media article to be read by an appropriate drive or via an appropriate connection, such as a DVD or flash memory device) to enable the computer-readable medium to execute or otherwise use or provide the contents to perform at least some of the described techniques. Some or all of the components and/or data structures may be stored on tangible, non-transitory storage mediums. Some or all of the system components and data structures may also be stored as data signals (e.g., by being encoded as part of a carrier wave or included as part of an analog or digital propagated signal) on a variety of computer-readable transmission mediums, which are then transmitted, including across wireless-based and wired/cable-based mediums, and may take a variety of forms (e.g., as part of a single or multiplexed analog signal, or as multiple discrete digital packets or frames). computer program products may also take other forms in other embodiments. Accordingly, embodiments of this disclosure may be practiced with other computer system configurations.

As described in FIGS. 1 and 2, there are different client applications that can be used to interact with ADMS Server 1010. These client application can be implemented using a computing system (not shown) similar to that described with respect to FIG. 10. Note that one or more general purpose virtual or physical computing systems suitably instructed or a special purpose computing system may be used to implement an ADMS client. However, just because it is possible to implement the Aesthetic Delta Measurement System on a general purpose computing system does not mean that the techniques themselves or the operations required to implement the techniques are conventional or well known.

All of the above U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 63/145,463, entitled “METHOD AND SYSTEM FOR QUANTIFYING AND VISUALIZING CHANGES OVER TIME TO AESTHETIC HEALTH AND WELLNESS,” filed Feb. 3, 2021, is incorporated herein by reference, in its entirety.

From the foregoing it will be appreciated that, although specific embodiments have been described herein for purposes of illustration, various modifications may be made without deviating from the spirit and scope of the invention. For example, the methods and systems discussed herein are applicable to other architectures. Also, the methods and systems discussed herein are applicable to differing protocols, communication media (optical, wireless, cable, etc.) and devices (such as wireless handsets, electronic organizers, personal digital assistants, portable email machines, game machines, pagers, navigation devices such as GPS receivers, etc.).

1. A computing system comprising:
a memory;
a computer processor;
a client interface, stored in the memory and executing under control of the processor, the interface configured to automatically:
present a display of a plurality of images as a batch of images;
present a visual guide and a scale, wherein each point on the scale corresponds to a corresponding image on the visual guide and wherein each subsequent image on the visual guide starting with the first represents a differing degree a determined characteristic is present in the visual guide image;
present a display of instructions for assigning an objective score to each of the plurality of images based upon the visual guide and the scale; and
for each image of the plurality of images in the batch of images,
receive an indication from a user of an assignment of a corresponding position on the scale to the image; and
forwarding annotated data regarding the image and its scalar value based upon the corresponding position on the scale to a server configured to receive and store aesthetic data.

2. The computing system of claim 1 wherein the client interface is configured to forward the annotation data to a server configured to consume the annotated data as training data, validation data, or test data in a machine learning model.

3. The computing system of claim 1 wherein the plurality of images are images of facial features.

4. The computing system of claim 1 wherein the visual guide and scale correspond to facial aging measurements.

5. The computing system of claim 1 wherein the indication from the user of an assignment of a corresponding position on the scale to the image is performed by dragging the image to a corresponding position on the scale using a slider user interface control.

6. The computing system of claim 1, further comprising an administrator interface configured to detect a user fraudulently scoring images.

7. The computing system of claim 6 wherein the administrator interface is configured to detect a user fraudulently scoring images by detecting whether the same score has been assigned to all images in a single batch.

8. The computing system of claim 1 wherein the client interface is configured to integrate functions of a crowd sourcing software system and wherein the results of participant users assigning scalar positions to each image are compared with ground truth data manually assigned to that image to allow reassignment of ground truth values.

9. A computing system of comprising:
a memory;
a computer processor;
a plurality of images, wherein the number of images exceeds thousands of images,
a client interface, stored in the memory and executing under control of the processor, the second interface configured to automatically:
present a display of a plurality of images in a batch of images for pairwise comparison wherein first and second images are presented along with an indicator of equality, and wherein the batch of images for pairwise comparison is a small subset of the plurality of images;
presenting a set of instructions to a user for determining which of the first and second images should be selected over the other of the first and second images;
receiving an indication from the user of a selection of either of the first or second image or of the indicator of equality; and
forwarding the indicated image or indication of equality to a server for dynamically ranking the image to a position among the entirety of the plurality of images.

10. The computing system of claim 9 wherein the annotated data regarding the image and its scalar value and/or the indicated image or indication of equality for dynamically ranking the image is received from crowd sourced data.

11. The computing system of claim 9 wherein the annotated data regarding the image and its scalar value and/or the indicated image or indication of equality for dynamically ranking the image is received from a participant user having associated images of the participant user that are scored by the participant user.

12. The computing system of claim 9 wherein the batch of images for pairwise comparison is for comparing glabellar lines and/or forehead lines.

13. The computing system of claim 9 wherein the indicated image for dynamically ranking is forwarded to a server configured for consumption as training data, validation data, or test data in a machine learning model.

14. A computer-implemented method for scaling and/or ranking visual aesthetic human body related data using pairwise comparisons of images comprising:
presenting a display of images in a batch of images for pairwise comparison wherein first and second images are presented along with an indicator of equality, and wherein the batch of images for pairwise comparison is a small subset of the plurality of images;
presenting a set of instructions to a user determining which of the first and second images should be selected over the other of the first and second images;
receiving an indication from the user of a selection of either of the first or second images or of the indicator of equality; and
forwarding the indicated image or indication of equality to a server for dynamically ranking the image to a position among the entirety of the plurality of images.

15. A computer-readable storage medium containing instructions for controlling a computer processor, when executed, to scale and/or rank visual aesthetic human body related data using pairwise comparisons of images by performing a method comprising:
presenting a display of a plurality of images in a batch of images for pairwise comparison wherein first and second images are presented along with an indicator of equality, and wherein the batch of images for pairwise comparison is a small subset of the plurality of images;
presenting a set of instructions to a user determining which of the first and second images should be selected over the other of the first and second images;
receiving an indication from the user of a selection of the first or second image or of the indicator of equality; and
forwarding the indicated image or indication of equality to a server for dynamically ranking the image to position among the entirety of the plurality of images.

16. The computing system of claim 1 wherein the visual guide and scale correspond to forehead lines and/or glabellar lines.

17. The method of claim 14 wherein the indicated image or indication of equality for dynamically ranking the image is received from crowd sourced data.

18. The method of claim 14 wherein the indicated image or indication of equality for dynamically ranking the image is received from a participant user having associated images of the participant user that are scored by the participant user.

19. The computer-readable storage medium of claim 15 wherein the indicated image or indication of equality for dynamically ranking the image is received from crowd sourced data.

20. The computer-readable storage medium of claim 15 wherein the indicated image or indication of equality for dynamically ranking the image is received from a participant user having associated images of the participant user that are scored by the participant user.

21. The computer-readable storage medium of claim 15 wherein the batch of images for pairwise comparison is for comparing glabellar lines and/or forehead lines.

22. The computer-readable storage medium of claim 15 wherein the indicated image for dynamically ranking is forwarded to a server configured for consumption as training data, validation data, or test data in a machine learning model.
